(19) United States
(12) Patent Application Publication
(10) Pub. No.: US 2007/0077289 A1
(43) Pub. Date: Apr. 5, 2007

(54) Cream containing interferon encapsulated with liposome

(75) Inventors: Yan Wang, Shenzhen City (CN); Xiangdong Chai, Shenzhen City (CN); Yunfu Li, Shenzhen City (CN); Baoke Dong, Shenzhen City (CN); Xuetao Zhang, Shenzhen City (CN)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: SHENZHEN NEPTUNUS INTERLONG BIO-TECHNIQUE HOLDINGS, Shenzhen City (CN)

(21) Appl. No.: 10/597,155

(22) PCT Filed: Jul. 13, 2004
(86) PCT No.: PCT/CN04/00803

§ 371 Date: Jul. 13, 2006

(30) Foreign Application Priority Data
Jan. 16, 2004 (CN) 200410015169.0

(51) Int. Cl. A61K 9/127; A61K 38/21

(52) U.S. Cl. 424/450; 424/85.4; 424/85.5; 424/85.6; 424/85.7; 977/907

(57) ABSTRACT

This invention relates to a cream containing interferon encapsulated with liposome, which comprises IFN liposome and cream substrate. The advantage of the cream of the present invention is high efficiency of encapsulation, stable production technique, good homogeneity of products, stable efficacy and low rate of leakage. In order to improve the capability of encapsulation of liposome and the activity and efficacy of IFN, IFN liposome and substrate are utilized to prepare a form of cream which cures those skin disease infected by virus, such as herpes zoster, herpetic stomatitis, verruca, condyloma acuminata, mollusoum contagiosum, herpes genitalis, verrca planae, verruca vulgaris, genital ulcer, oral ulcer and pruritus.

### FIELD OF THE INVENTION

This invention relates to a new form of Interferon, particularly relates to a cream containing interferon encapsulated with liposome.

### BACKGROUND OF THE INVENTION

Interferon (hereinafter abbreviated as IFN) is widely used as anti-virus, anti-proliferation and immunomodulatory drug, whose dosage forms are mainly injection and external use. Besides, IFN administered externally can directly act on sites of lesion, which is convenient to use. However, it is found that the problem of both maintaining activity and absorption through skin should be solved during application. In order to keep the stability of biological activity and improve the absorption through skin to reach the goal of treatment, a better method is to encapsulate IFN in liposome and then prepare the corresponding dosage form for external use. But there exist many defects in the existing techniques of encapsulating IFN in liposome. For example, in foreign references, it was merely reported that liposome was used as carrier of drugs administered externally to encapsulate IFN, but IFN liposome could not tightly bind to skin or mucosa after application to function, which greatly lowered the effect of treatment. For another example, Chinese patent No. 97109122.6 provides a kind of preventive drug containing liposome, and lodophors which decrease biological activity of IFN and influence effectiveness of IFN due to the damaged to IFN itself and meanwhile to liposome owing to high oxidization. In addition, Chinese patent No.97109123.4 provides a kind of IFN-encapsulated liposome gel which is a combination of gel and IFN liposome, with IFN converted into a form of powder after dehydration and subsequently into gel water solution after dissolving thoroughly in water prior to application, and then used as drug for external use. Meanwhile, it is necessary to mix excipient with IFN liposome at 0-4° C. during preparation of this kind of IFN-encapsulated liposome gel, which is turned into a particle powder form of IFN-encapsulated liposome gel after dehydration. Therefore, the gel in this invention possesses the following defects: 1. complicated preparation techniques, especially the frozen-dry process which significantly decreases IFN biological activity; 2. inconvenient to use due to the gel must be dissolved in water as using the drug for smearing.

### SUMMARY OF THE INVENTION

The object of this invention is to provide a kind of cream containing IFN that are superior to the previously known forms of IFN with regard to having effect of decelerated release and controlled release on IFN, stable effectiveness and easy to be evenly smeared on skin mucosa, excellent adhesive ability, zero stimulation, easy to be absorbed through skin and convenient to use. Besides, the substrate formula of the present invention conduces to stabilize the IFN liposome of cream.

Accordingly, in one aspect the present invention provides a kind of IFN encapsulated with liposome (hereinafter named as IFN liposome). The IFN is either natural or recombinant, and currently all the common forms of IFN is suitable for use in this invention, such as IFN-α, β and γ, preferably IFN-α, which is more suitable for external use drugs due to its anti-virus activity. In a preferred embodiment of the invention, IFN-α2b, having high ratio of activity, high anti-virus activity and low rate of counteracting antibody, is utilized for preparation of the cream.

Encapsulating IFN with liposome may bring the IFN encapsulated thereof to be decelerated release and controlled release and meanwhile improves the stability of IFN, and can boost absorption of IFN through skin because the lipid bilayer of the liposome enormously resembles biological membrane and is apt to be absorbed owing to compatibility with tissues. Various kinds of liposome can be used to encapsulate IFN in this invention, and in a preferred embodiment of the invention, membrane material and anti-oxidization agent are used as materials for preparing the IFN liposome, wherein the membrane material can be selected from the group consisting of phosphatidyl choline, soya lecithin, cephalin, lecithin, cholesterol, stearic amide and mixtures thereof, preferably the mixtures of soya lecithin, cholesterol and stearicamide; and vitamin E is used as anti-oxidization agent.

During preparation of IFN liposome, rotate and evaporate both membrane materials and anti-oxidization agent dissolved in CH₂Cl₂ in a certain proportion, then add IFN solution of an anticipated concentration after drying the organic solvent and keep rotating until IFN liposome suspension comes into being, and finally collect the target part to acquire IFN liposome by sonication and through subsequent gel filtration. In a particular embodiment of this invention, the formula of membrane material and anti-oxidation agent for preparation of liposome is the following: phosphatide 65-90 parts by weight ; cholesterol 5-30 parts by weight; stearic amide 0.5-5 parts by weight; and vitamin E 0.2-2 parts by weight, preferably the weight ratio of soya lecithin: cholesterol: stearic amide: vitamin E is 80:18:1:1. Furthermore, the ratio of IFN biological activity to the volume of liposome solution is 10⁵-10⁸ IU: 1 ml, and the encapsulation rate of IFN liposome is no less than 80%.

In another aspect, the present invention provides a cream containing IFN encapsulated with liposome. The cream of the invention can be prepared by mixing thoroughly the IFN liposome with cream substrate. The cream substrate of the present invention comprises the following components: excipient 200-300 parts by weight; emulsifier 10-30 parts by weight; stabilizer 5-25 parts by weight; and antiseptic 0.5-1 parts by weight, wherein the excipient can be selected from the group consisting of glycerin, liquid paraffin, stearic acid, glyceryl monostearate, octadecanal, white vaseline, yellow vaseline, lanolin and mixtures thereof, preferably the mixtures of glycerin, glyceryl monostearate and white vaseline; the stabilizer can be selected from the group consisting of mannit, sucrose, β-cyclodextrin, dextran 40, trehalose, ethyl lactate and mixtures thereof, preferably the mixtures of dextran 40 and ethyl lactate; and the antiseptic can be selected from the group consisting of methyl p-hydroxybenzoate, ethyl p-hydroxybenzoate, propyl p-hydroxybenzoate and mixtures thereof, preferably ethyl p-hydroxybenzoate.

In the cream formulation of this invention, emulsifier plays a role not only in modulating the adhesive ability and the emulsification efficiency, forming a even dosage form through emulsification of both oil phase and water phase which are not dissolved in each other at all, but also in stabilizing liposome, decreasing leakage of IFN out of liposome and improving the stability of IFN liposome. In the embodiments of this invention, any kind among polysorbate 20, polysorbate 60, polysorbate 80, span 80 and sodium dodecyl sulfate can be used as emulsifier, preferably polysorbate 80 which significantly improves the stability of IFN liposome and whose protective concentration ranges between 2.0%-5.0% by weight (polysorbate 80: cream substrate). Furthermore, in a preferred embodiment, the components of cream substrate are glycerin: glyceryl monostearate: white vaseline: polysorbate 80: dextran 40: ethyl lactate: ethyl p-hydroxybenzoate in a ratio of 20:20:5:3:1:1:0.1 by weight.

During preparation of the cream of the invention, first of all, prepare the IFN liposome solution and sterilize it for stock. Second of all, according to the formula of substrate, respectively dissolve or melt oil phase, including excipient and emulsifier, and water phase, including stabilizer, antiseptic and distilled water, sterilize them or remove bacteria after even agitation. Ultimately mix oil phase with water phase together to prepare cream substrate, add IFN liposome solution to cream substrate in an anticipated concentration, stir it until even and separately load it, which is the accomplishment of the cream.

During preparation, the ratio of volume of IFN liposome solution to weight of substrate is 5-20:80-95 (ml/g), and the ratio of IFN biological activity to weight of cream substrate is 5×10³ IU-5×10⁶ IU:1 g, preferably 5×10⁴ IU:1 g.

### DETAILED DESCRIPTION OF THE INVENTION

The invention will be further described in detail in the following concrete examples. However, this should not be so understood that the invention theme is only limited in the following examples.

Materials:

IFN-α: according to rule and regulation of biological product in China, 2000 edition, prepare IFN-α through bacterial fermentation and column chromatography system.

Other reagents and material are purchased commercially.

### EXAMPLE 1

### Preparation of Cream Containing IFN Encapsulated with Liposome

1. Preparation of IFN liposome

\textcircled{1} dilution of IFN initial solution

dilute IFN initial solution with PBS containing 0.8% human blood albumin until IFN biological activity is 0.8×10⁷ IU/ml and sterilize the ultimate solution for stock.

\textcircled{2} Weight of membrane material

weigh phosphatidyl choline: choleterol: vitamin E in a 85:20:2:1 weight ratio, which are membrane material, definitely phosphatidyl choline, 4.38 g; cholesterol, 1.04 g; stearic amide, 0.1048 g; and vitamin E, 0.0516 g.

\textcircled{3} Rotary Evaporation

add CH₂Cl₂ up to 100 ml after completely dissolving all kinds of the above membrane material with CH₂Cl₂ in spherical flask, evaporate it at 40° C. at 1000 rpm under decreasing pressure in rotary evaporator, add 100 ml of diluted IFN solution and keep rotating after organic solvent is completely dried, which peel off lipid membrane on the wall of the flask, and then collect lipid suspension.

\textcircled{4} Sonicate

disperse the collected suspension with a bath sonicator.

\textcircled{5} Gel filtration

filter IFN liposome dispersed by sonicator through gel filtration column and collect 412 ml of solution of the first eluting peak, i.e. IFN liposome solution.

\textcircled{6} Filter and sterilize the passing-column IFN liposome through 0.22 u filter membrane and test it for stock.

2. Preparation of substrate

Precisely weigh liquid wax, glyceryl monostearate, white vasline, Span 80, mannit, ethyl p-hydroxybenzoate and propyl p-hydroxybenzoate at a ratio by weight.

\textcircled{1} oil phase

glyceryl monostearate, 4213 g; white vaseline, 1057 g; Span 80, 633 g and liquid wax, 4197 g, agitated thoroughly and mingled evenly.

\textcircled{2} water phase

mannit, 215 g; ethyl p-hydroxybenzoate, 21 g; propyl p-hydroxybenzoate, 21 g and distilled water, 8398 g, agitated thoroughly and mingled evenly.

\textcircled{3} melt both oil phase and water phase respectively, and mix oil phase with water phase to prepare substrate after filtration and sterilization,

3. Preparation of IFN-encapsulated liposome cream

Add IFN liposome to substrate at the ratio of IFN activity of IFN liposome to substrate, i.e. 5×10⁴ IU:1 g, and separately load it by 15 g/piece after agitated thoroughly and mingled evenly.

### EXAMPLE 2

### Preparation of Cream Containing IFN Encapsulated with Liposome

1. Preparation of IFN solution

\textcircled{1} dilution of IFN initial solution

dilute IFN initial solution with PBS containing 0.8% human blood albumin until IFN biological activity is 1.0×10⁷ IU/ml, and sterilize and filter the ultimate solution for stock.

\textcircled{2} weight of membrane material

weigh phosphatidyl choline: cholesterol: stearic amide: vitamin E in a 80:20:1:1 weight ratio, which are membrane material, concretely lecithin, 4.26 g; cholesterol, 1.06 g; stearic amide, 0.0532 g and vitamin E, 0.0532 g.

\textcircled{3} rotary evaporation

Add CH₂Cl₂ up to 100 ml after completely dissolving all kinds of the above membrane material with CH₂Cl₂ in spherical flask, evaporate it at 40° C. at 1000 rpm under decreasing pressure, and then add 100 ml of the diluted IFN solution and keep rotating after organic solvent is completely dried, which peels off liposome membrane on the wall of the flask and then collect liposome suspension.

\textcircled{4} sonication

disperse the collected suspension with a bath sonicator.

\textcircled{5} gel filtration

filter IFN liposome solution dispersed by sonicate through gel column and collect 412 ml of solution of the first eluting peak, i.e. IFN liposome solution.

\textcircled{6} Filter and sterilize the passing-column liposome solution through 0.22 u filteration membrane and test it for stock.

2. Preparation of substrate

Precisely weigh liquid wax, glyceryl monostearate, white vaseline, polysorbate 20, dextran 40, mannit and ethyl p-hydroxybenzoate at a ratio of weight.

\textcircled{1} oil phase

glyceryl monostearate, 4196 g; white vaseline, 1047 g; polysorbate 20, 631 g and liquid wax, 4215 g, agitated thoroughly and mingled evenly.

\textcircled{2} water phase

mannit, 208 g; dextran 40, 214 g; propyl p-hydroxybenzoate, 21 g and distilled water, 8414 g, agitated thoroughly and mingled evenly.

3. The method of preparation is similar to that of Example 1.

### EXAMPLE 3

### Preparation of Cream Containing IFN Encapsulated With Liposome

1. Components:

Material for preparation of IFN liposome:

IFN-α2b initial solution is prepared through bacteria fermentation and column chromatography system, whose biological activity is not less than 1.0×10⁸ IU/ml, according to rule and regulation of biological product in China, 2000 edition;

Raw material and formula for preparation of liposome are similar to those in Example 2.

2. Method of preparation

\textcircled{1}Preparation of IFN liposome

the production method is similar to that in Example 2 except that soya lecithin replaces lecithin.

\textcircled{2} preparation of substrate

Precisely weigh glycerin: glyceryl monostearate: white vaseline: polysorbate 80: dextran 40: ethyl lactate: ethyl p-hydroxybenzoate in a 20:20:5:3:1:0.1 weight ratio.

\textcircled{1} oil phase

glyceryl monostearate, 4200 g; white vaseline, 1050 g and polysorbate 80, 630 g, agitated thoroughly and mingled evenly.

\textcircled{2} water phase

glycerin, 4200 g; dextran 40, 210 g; ethyl p-hydroxybenzoate, 21 g; ethyl lactate, 210 g and distilled water, 8400 g, agitated thoroughly and mingled evenly.

\textcircled{3} the method of preparation is similar to that of Example 1.

3. Preparation of cream

the method of preparation is similar to that of Example 1.

### EXAMPLE 4

### Preparation of cream containing IFN encapsulated with liposome

1. Preparation of IFN liposome

The method of preparation is similar to that of Example 3.

2. Preparation of substrate

precisely weigh glycerin, stearic acid, white vaseline, polysorbate 80, maltose, ethyl lactate and ethyl p-hydroxybenzoate at a ratio of weight.

\textcircled{1} oil phase

stearic acid, 4203 g; white vaseline, 1047 g and polysorbate 80,633 g, agitated thoroughly and mingled evenly.

\textcircled{2} water phase

glycerin, 4201 g; β-cyclodextrin, 213 g; ethyl p-hydroxybenzoate, 21 g; ethyl lactate, 206 g and distilled water, 8397 g, agitated thoroughly and mingled evenly.

the method of agitation is similar to that of Example 1.

\textcircled{3} the method of preparation is similar to that of Example 1.

3.Production of cream

The method of preparation is similar to that of Example 1.

### EXAMPLE 5

### Preparation of Cream Containing IFN Encapsulated with Liposome

1. Preparation of IFN liposome

the method of preparation is similar to that of Example 3 except that soya lecithin of membrane material of liposome is substituted for cephalin.

2. Preparation of substrate

precisely weigh glycerin, octadecanal, glyceryl monostearate, white vasline, sodium dodecyl sulfate, dextran 40, ethyl lactate and ethyl p-hydroxybenzoate at a ratio of weight.

\textcircled{1} oil phase

glyceryl monostearate, 4212 g; octadecanal, 2095 g; white vasline, 1051 g and sodium dodecyl sulfate, 628 g, agitated thoroughly and mingled evenly.

\textcircled{2} water phase

glycerin, 2110 g; ethyl p-hydroxybenzoate, 21 g; ethyl lactate, 213 g and distilled water, 8402 g, agitated thoroughly and mingled evenly.

\textcircled{3} the method of preparation is similar to that of Example 1.

3. Production of cream

The method of preparation is similar to that of Example 1.

### EXAMPLE 6

### Pharmacological and Toxicological Experiments of Cream Containing IFN Encapsulated with Liposome

1. Pharmacological experiment

(1) material: Guinea pigs, white mice and cats, purchased; cream containing IFN encapsulated with liposome, sample prepared according to Example 3.

(2) Method

\textcircled{1} cream containing recombinant IFN-α2b encapsulated with liposome of a concentration of 0.6×10⁴, 1.0×10⁴ and 10.0×10⁴ IU per animal respectively, is smeared on affected surface of Guinea pigs twice a day for consecutive 7 days and observe whether it significantly inhibits the experimental viral herpes simplex of Guinea pig the extent of inhabitation increases with an increasing dosage.

\textcircled{2} the cream of a concentration of 0.9×10⁴, 1.5×10⁴ and 15.0×10⁴ IU/per animal respectively is smeared on broken skin of white mice and observe whether the drug has an impact on self-dominated activity of these mice.

\textcircled{3} the cream of a concentration of 0.3×10⁵, 0.5×10⁵ and 5.0×10⁵ IU/per animal respectively is smeared on skin of anesthetized cats and observe whether the drug has an impact on their blood pressure, heart rate, breath frequency, breath depth and electrocardiogram.

(3) Result

The drug significantly inhibits the experimental viral herpes simplex of Guinea pigs, and the extent of inhibitation is rising with an increasing dosage and has significant effect neither on self-dominated activity of little white rats nor on blood pressure, heart beat, breath frequency, breath depth and electrocardiogram of anesthetized cats.

2. Acute toxicity experiment of rats

(1) materials

rats, purchased; cream containing IFN encapsulated with liposome, sample prepared in Example 3.

(2) method

smear the cream on broken surface of rats by a maximal dosage of 2.78×10⁴ IU/Kg body weight and observe them for consecutive 7 days and record both abnormal phenomenon and death toll.

(3) result

There don't occur death and abnormal phenomenon within 7 days, and the dosage is equivalent to 1.1×10⁴ times clinical dosage, so clinical application is very secure.

3. Long-term toxicity experiment

(1) materials

rats, purchased; cream containing IFN encapsulated with liposome, sample in Example 3, while the control is liposome cream free of IFN prepared in accordance with Example 3.

(2) method

administrate the cream by a dosage of 2.78×10⁷ IU/per animal on broken skin once a day for consecutive 28 days ( equivalent to 4 times anticipated clinical cycle time) and compared to the control group, observe activity, weight, consumed food, drunk water, blood routine test, blood biochemical parameter and organ index of rats and conduct histopathological examination and record abnormal phenomenon and death toll.

(3) result

Compared to the control group, abnormal phenomena do not occur concerned about all parameters observed, neither do those mentioned-above parameters observed for 14 days without imposing medicine after consecutive 28 days of administration beforehand.

### EXAMPLE 6

### Security Test Experiment of Cream Containing IFN Encapsulated with Liposome

1. Skin allergy experiment

(1) material:

Guinea pigs, purchased; cream containing IFN encapsulated with liposome, sample prepared according to Example 3, whose biological activity is 5.0×10⁴ IU/g; negative control, sample free of IFN prepared according to Example 3; positive control, 2,4-chloro dinitro benzene, purchased.

(2) method

Guinea pigs are divided into 3 groups, namely tested group, negative control group and positive control group, after shedding hair on the back and these 3 groups are administrated with the cream containing IFN liposome of a dosage of 0.2 g/time, additionally negative control group of a dosage of 0.2 g/time and positive control group of 0.1% of a dosage of 0.2 ml/time respectively for sensitization contact, and then respectively repeat the above steps in day 7 and day 14 and after another 14 days, smear the same dosage o f the tested drug, negative control and positive control on sites of action for stimulation contact, remove them after 6 hours and immediately observe skin allergic situation and subsequently observe it once again in hour 24, 48 and 72 and record reaction phenomena.

(3) result

redness, swelling, necrosis and other allergic response are not observed in the drug group and negative control group, however significant allergic response are observed in positive control group.

2. Skin stimulation experiment

(1) material

Guinea pigs, purchased; cream containing IFN encapsulated with liposome, sample prepared according to Example 3, whose biological activity is 5.0×10⁴ IU/g.

(2) method

Conduct experiment according to the method of page 205 of “assemble of guidance principle of new medicine (western medicine) clinical study” edited by Pharmaceutical administration bureau of ministry of health, P.R.C in July, 1993 and page 262 of “study and application of new Chinese traditional medicine” edited by Wang Beiyin, published by Chinese traditional medicine press of China.

(3) result

the cream of a concentration of 5.0×10⁴ IU/g is smeared on skin at a dosage of 1.0 g/time for consecutive 7 days and has not stimulative response to not only broken skin but also healthy skin of Guinea pigs.

### EXAMPLE 7

### Main Pharmacodynamics Experiment of Cream Containing IFN Encapsulated with Liposome

Effect on viral herpes simplex

(1) materials

Guinea pigs, purchased; cream containing IFN encapsulated with liposome, 3 dosages of drug prepared according to Example 3, whose biological activity is 0.3×10⁴ IU/g, 0.5×10⁴ IU/g and 5.0×10⁴ IU/g respectively; positive control, Acyclovor ointment, produced by Shanghai GE pharmaceutical holdings Co. Ltd; virus, herpes simplex virus provided by teaching and research room of pathogenic biology of basic medical school of Jilin University.

(2) method

Conduct experimcnt according to the method of page 169 of Assemble of guidance principle of new medicine (western medicine) clinical study, edited by Pharmaceutical administration bureau of ministry of health, P.R.C in July, 1993.

(3) result

(1) There occur a hard papule, big bubble or irregular dispersing water blister on sites of inoculation 6-8 days after viral inoculation, which suggests the model is successful.

(2) Compared to model groups, each drug group recovers 1-2 days after administration, whereas the extent of recovery doesn't differ significantly, which suggests the cream containing IFN encapsulated with liposome significantly shortens the duration of viral herpes simplex, and accelerates scab and recovery of water blister.

(3) Compared to positive control group, the cream containing IFN encapsulated with liposome deposits on sick and damaged sites for a longer time and possesses a stronger biological activity against viral herpes simplex.

### EXAMPLE 8

### Experiment of Stability of Cream Containing IFN Encapsulated with Liposome Contributed to by Polysorbate 80

materials:

polysorbate 80, pharmaceutical grade, produced by Chaoneng Industrial Co. Ltd, Zhaoqing, Guangdong Province;

Initial solution of IFN liposome, prepared according to the mentioned-above method, whose IFN biological activity is 6.2×10⁶ IU/ml;

PBS, phosphate buffer solution, whose concentration is 0.1M and pH 7.2;

lysis reagent, PBS composed of 0.04% of Triton X-100;

raster spectrophotometry meter 772, produced by Shanghai No.3 analysis instrument factory;

upside-down microscope, type CK2, produced by Olympus Co., Japan.

Method:

dilute IFN liposome 60 times with PBS until its activity is 1.0×10⁴ IU/ml and polysorbate 80 with PBS until their concentration are 1%, 2%, 5% and 10% respectively; fill 6 tubes with 2 ml of the diluted IFN liposome respectively, which are marked No.1-No.6 for stock and subsequently No.1-No.4 tubes with 2 ml of the various diluted polysorbate solution respectively until the ultimate concentrations of polysorbate 80 are 0.5%, 1%, 2.5% and 5% respectively and No.5 tube with 2 ml of PBS as negative control and No.6 tube with 2 ml of lysis reagent as positive control respectively, and thereof the ultimate concentration of TritonX 100 in NO.6 is 0.02%; all samples are heated in water bath at 40° C. for 5 minutes, whose OD value is measured by Raster spectrophotometry meter 772 under measure wavelength of 600 nm; measure biological activity of samples separately picked from those bathed; and finally adopt the method of CPE restraint method to measure biological activity of IFN.

Result:

| Specimen | Ultimate concentration of | | Biological |
|---|---|---|---|
| No. | polysorbate 80(%) | OD value | activity (IU/ml) |
| 1 | 0.5 | 0.210 | 1.98 × 10⁴ |
| 2 | 1 | 0.208 | 1.18 × 10⁴ |
| 3 | 2.5 | 0.203 | 0.65 × 10⁴ |
| 4 | 5 | 0.212 | 0.69 × 10⁴ |
| 5 | negative control | 0.211 | 1.76 × 10⁴ |
| 6 | positive control | 0.012 | 4.05 × 10⁴ |

### CONCLUSION

From those OD values, it is concluded that those OD values of liposome solution of various concentration of polysorbate 80 are almost in line with that of negative control, which reveals that polysorbate 80 does not lysis liposome, whereas the OD value of positive control is significantly low, which illustrates PBS solution of ultimate concentration of 0.02% Triton X-100 can lysis liposome of low concentration.

From those biological activity, it is also concluded that biological activity of unbound IFN in liposome solution decreases with an increasing concentration of polysrobate 80, however biological activity of unbound IFN in liposome solution containing 2.5%-5.0% of polysorbate 80 (polysorbate 80: substrate, w/w) does not alter significantly, which indicates protective concentration of polysorbate 80 on liposome is 2.5% or so.

Anyway, not greater than 5% polysorbate 80 does not destroy but stablize liposome, namely decrease leakage of liposome IFN.

### INDUSTRIAL APPLICATION

The cream containing IFN encapsulated with liposome of this invention is of high encapsulation rate, stable production technique, good homogeneity of product, stable efficacy and low leakage rate. Both IFN liposome and substrate are utilized to prepare cream dosage form which improves the capability of encapsulation of liposome and the biological activity and efficacy of IFN, and meanwhile which is of simple preparation and convenient usage. Polysorbate 80 is used as emulsifier in substrate which does not destroy but stabilize liposome, and accordingly decreases leakage of IFN and improves the stability of IFN liposome and the efficacy of IFN. The cream containing recombinant IFN-α2b encapsulated with liposome of this invention can cure those skin diseases infected by viruses, such as herpes zoster, herpetic stomatitis, verruca, condyloma acuminata, mollusoum contagiosum, genital herpes, verruca planae, genital ulcer, oral ulcer and pruritus, and meanwhile of evenly smear on skin mucosa, good adhesive ability, zero stimulation, good absorption through skin and convenient usage.

The above detailed description of this invention does not confine it, and according to this invention, technicians of this field can make all kinds of change and transformation which belong to the range as defined in claims of this invention as long as they don't deviate the spirit of this invention.

What is claimed is:

1. A cream containing interferon encapsulated with liposome, comprising:
an interferon encapsulated with liposome, and a cream substrate wherein:
the content of interferon is 1.0×10⁴-5×10⁸ IU/g cream at the ratio of biological activity of interferon to weight of cream;
the ratio of biological activity of interferon to weight of cream substrate is 5×10³-5×10⁶ IU:1 g;
and the ratio of biological activity of interferon encapsulated with liposome to total volume of liposome is 1.0×10⁵-5×10⁸ IU: 1 ml in the case of greater than 80% efficiency of encapsulation.

2. (canceled)

3. The cream containing interferon encapsulated with liposome according to claim 1, wherein the ratio of biological activity of interferon to the weight of cream substrate is 5×10⁴ IU:1 g.

4. The cream containing interferon encapsulated with liposome according to claim 1, wherein the liposome used for encapsulating interferon is made from the following components:
a membrane material, selected from the group consisting of phosphatidyl choline, soya lecithin, cephalin, lecithin, cholesterol, stearic amide and mixtures thereof, and
an antioxidant agent.

5. The cream containing interferon encapsulated with liposome according to claim 4, wherein said liposome is made from the following components:

| | |
|---|---|
| phosphatide | 65-90 parts by weight |
| cholesterol | 50-30 parts by weight |
| stearic amide | 0.5-5 parts by weight and |
| vitamin E | 0.2-2 parts by weight. |

6. The cream containing interferon encapsulated with liposome according to claim 5, wherein said liposome is made from the following components by weight:
phosphatide: cholesterol: stearic amide: vitamin E=80:18:1:1.

7. The cream containing interferon encapsulated with liposome according to claim 1, wherein said cream substrate comprising:

| | |
|---|---|
| excipient | 200-300 parts by weight |
| emulsifier | 10-30 parts by weight |
| stabilizer | 5-25 parts by weight; and |
| antiseptic | 0.5-1 parts by weight; |

and wherein:
said excipient is selected from the group consisting of glycerin, liquid paraffin, stearic acid, glyceryl monostearate, octadecyl alcohol, white Vaseline, yellow Vaseline, lanolin and mixtures thereof,
said emulsifier is selected from the group consisting of polysorbate 20, polysrobate 60, polysorbate 80, Span 80 and sodium dodecyl sulfate;
said stabilizer is selected from the group consisting of mannit, sucrose, maltose, dextran 40, trehalose, ethyllactate and mixtures thereof, and
said antiseptic is selected from the group consisting of methyl p-hydroxybenzoate, ethyl p-hydroxybenzoate, propyl p-hydroxybenzoate and mixtures thereof

8. The cream containing interferon encapsulated with liposome according to claim 7, wherein the emulsifier is polysorbate 80.

9. The cream containing interferon encapsulated with liposome according to claim 8, wherein the ratio of polysorbate 80 to cream substrate is 2.0%-5.0% by weight.

10. The cream containing interferon encapsulated with liposome according to claim 9, wherein the cream substrate comprising:
white Vaseline: polysorbate 80: dexcran 40: ethyl lactate: ethyl p-hydroxybenzoate=20:20:5:3:1:1:0.1 by weight.

11. The cream containing interferon encapsulated with liposome according to claim 1, wherein said interferon is natural IFN or recombinant human IFN-α, β or γ.

12. The cream containing interferon encapsulated with liposome according to claim 11, wherein said interferon is IFN α-2a, IFN α-1b or IFN α-2b.
